(19) United States
(12) Patent Application Publication
(10) Pub. No.: US 2006/0057716 A1
(43) Pub. Date: Mar. 16, 2006

(54) Human malignant cystosarcoma phyllodes derived mouse cell line

(76) Inventors: Louis Wing-Cheong Chow, Hong Kong (HK); Tjing Yung Loo, Hong Kong (HK)

Correspondence Address: Robert D. Katz, Esq.;Cooper & Dunham LLP, 1185 Avenue of the Americas, New York, NY, 10036, US

(21) Appl. No.: 10/942,068

(22) Filed: Sep. 15, 2004

(51) Int. Cl. C12N 5/06; C12N 15/87

(52) U.S. Cl. 435/354; 435/455

(57) ABSTRACT

A new mouse cell line, MCP-5, derived from human malignant cystosarcoma phyllodes was established. This invention provides methods for establishing MCP-5. MCP-5 may serve as both in vitro and in vivo models for studying pathogenesis and experimental therapy of human malignant cystosarcoma.

### BACKGROUND OF THE INVENTION

Cystosarcoma phyllodes are rare breast tumors characterized by proliferation of both stromal and epithelial elements [1]. They present as masses, often large ones [2], in middle-aged women, most commonly in the fourth and fifth decades of life [3], only exceptionally in pre-puberty or adolescence [4-6].

Phyllodes tumors of the female breast represent less than 0.5% of all breast neoplasms [7] and about 2.5% of fibro-epithelial tumors [8]. This disease shows a regional predilection that may be attributable to racial susceptibility, dietary habits and exposure to causative factors, as it seems to exhibit some different variations in the Asian population from the Western patients [9]. In Chua's study, they have also highlighted the relatively high incidence of phyllodes tumors among Asian women [9].

In 1838, Johannes Muller coined the term “cystosarcoma phyllodes”, emphasizing its benign nature and differentiation from adenocarcinoma [10]. In 1931, the occasional malignant nature of this tumor was recognized [11]. The tumors were classified into benign, borderline and malignant categories, using the criteria of stromal cellularity, margins, mitotic index and cellular atypia [12-14]. Chua et al. classified malignant phyllodes as having high cellularity, pleomorphism and anaplasia, infiltrative margins and mitoses>10/10 HPF [9]. Metastatic spread of cystosarcoma occurs, but infrequently, ranging from 3% to 12% [15]. Local recurrence is more often seen [16]. The incidence of this phenomenon has ranged from 7.5% to 58.8% [17,18].

Anticancer drug screening trials have been conducted by the pharmaceutical industry for many years by the use of tumor cell lines as in vitro models. Drugs that show anticancer activity in vitro are subsequently tested against a corresponding in vivo tumor model. However, not all in vitro models genetically or pathologically match the in vivo systems. The present inventors have successfully established a cell line, MCP-5, that is able to grow in both in vitro and in vivo systems. Thus, one object of the present invention is the development of a cell line (MCP-5) that is suitable for both in vitro and in vivo investigations.

### SUMMARY OF THE INVENTION

This invention provides a cell line of mouse origin derived from a human malignant cystosarcoma phyllodes tumor designated MCP-5.

This invention further provides a method for establishing a cell line of mouse origin derived from a human malignant cystosarcoma phyllodes tumor, comprising transplanting the human malignant cystosarcoma phyllodes tumor to a recipient nude mouse; removing the tumor from the recipient nude mouse after allowing sufficient time for tumor growth; and culturing the cells of the tumor in a suitable culture medium, thereby establishing a cell line of mouse origin derived from a human malignant cystosarcoma phyllodes tumor.

### BRIEF DESCRIPTION OF THE DRAWINGS

FIG. 1.

Tumor in nude mouse from transplant of clinical malignant cystosarcoma phyllodes.

FIG. 2.

Histology of malignant cystosarcoma phyllodes: a) original tumor from patient, b) tumor from nude mouse induced by cultured cells and c) xenograft cell line MCP-5 showing spindle cells growing on glass slide. Hematoxylin-eosin staining. Magnification 40×.

FIG. 3.

In vitro growth of cells in D-MEM/F-12.

FIG. 4.

Immunohistochemical staining for VEGF in xenograft cell line.

FIG. 5.

Electron micrograph of cultured MCP-5 cell line showing exceptionally large nucleus leading to a lower nucleus to cytoplasm ratio with hyper-chromatographic nucleolus also shown.

FIG. 6.

Representative karyotype of MCP-5 cell line.

### DETAILED DESCRIPTION OF THE INVENTION

### DEFINITIONS

As used in this application, except as otherwise expressly provided herein, each of the following terms shall have the meaning set forth below.

As used herein, “mastectomy” shall mean the surgical removal of one or both breasts to remove a malignant tumor. A mastectomy is sometimes followed by flap reconstruction described herein.

As used herein, “flap reconstruction” shall mean the use of pedicle flap to cover injury sites.

As used herein, “myoepithelial” shall mean the features of both muscular and epithelial tissues.

As used herein, “clinical tumor” shall mean any primary tumor mass clinically remove from the cancer patient.

As used herein, “transplantable tumor” shall mean any tumor mass transplanted onto any subject or supporting tissues.

As used herein, “nude mice” shall mean the mice that are homozygous for a recessive mutant gene designated nu. These mice are born without a thymus therefore lack immune responses mediated by T cells that are responsible for graft rejection.

As used herein, “xenograft” shall mean a graft of any tissues from foreign subjects.

As used herein, “cell line” shall mean any cell cultured in a quiescent or stationary phase which undergoes conversion to a state of unregulated growth in culture, resembling an in vitro tumor. It occurs spontaneously or through interaction with viruses, oncogenes, radiation, or drugs/chemicals.

As used herein, “immunohistochemistry” shall mean histochemical localization of immunoreactive substances using labeled antibodies as reagents.

As used herein, “electron microscopy” shall mean any form of microscopy in which the interactions of electrons with the specimens are used to provide information about the final structure of that specimen.

As used herein, “karyotyping” shall mean the process of analyzing and classifying the chromosome of a cell and preparing a karyotype diagram. Karyotyping is used in the diagnosis of certain chromosomal abnormalities.

Introduction

The object of this study was to establish a mouse cell line (MCP-5) derived from human cystosarcoma phyllodes. The transplanted phyllodes grew rapidly in nude mice after subcutaneous inoculation. The xenograft was cultured when a desirable tumor mass was reached. The cultured cells (MCP-5) derived from the xenograft described herein mimic the aggressive growth characteristics seen in the patient. The MCP-5 cell exhibited a steady growth rate during the time of culture and showed high tumorigenicity in nude mice. This invention provides both in vitro and in vivo models for the study of cystosarcoma phyllodes pathogenesis.

### Preferred Embodiments of the Invention

This invention provides a cell line of mouse origin derived from a human malignant cystosarcoma phyllodes tumor designated MCP-5. The MCP-5 cell line displays similar characteristics of a clinical cystosarcoma phyllodes tumor, which may comprise aggressiveness of tumor growth, tumorigenicity, or recurrence of tumors.

This invention further provides a method for establishing a cell line of mouse origin derived from a human malignant cystosarcoma phyllodes tumor, comprising transplanting the human malignant cystosarcoma phyllodes tumor to a recipient nude mouse; removing the tumor from the recipient nude mouse after allowing sufficient time for tumor growth; and culturing the cells of the tumor in a suitable culture medium, thereby establishing a cell line of mouse origin derived from a human malignant cystosarcoma phyllodes tumor. In one embodiment, the tumor may be transplanted by subcutaneous inoculation. In another embodiment, the suitable culture medium is D-MEM/F-12. In yet another embodiment, the suitable culture medium contains agents including, but not limited to, Dulbecco's Modified Eagle Medium with 15% Fetal Bovine Serum (FBS), 1 mM HEPES buffer, L-glutamine, pyridoxine hydrochloride, 100 U/ml penicillin, and 100 μg/ml streptomycin and 4 μg/ml insulin.

The above features and advantages of the present invention will be better understood with reference to the experimental details below. It should also be understood that the particular methods and cell lines illustrating the invention are exemplary only and are not to be regarded as limitations of the present invention.

Experimental Details

Background

The factors commonly used to establish a grade of phyllodes tumors include mitotic activity, status of the margins, and stromal atypia and overgrowth [9, 12-14, 20-22]. Even after pathologic evaluation of the tumor, determining the prognosis in patients with phyllodes tumors of the breast can be very difficult because it hardly correlates to natural history and histological appearance [23]. This points out the need to have a good model with which to study this unusual neoplasm and emphasizes that current criteria for establishing malignancy are inexact. This well-characterized cell line, although proven to be of mouse origin, will provide an important research tool with which to study biological, molecular, and endocrine regulation as well as experimental therapy of this rare tumor type.

Three malignant phyllodes cell lines have been established [7, 24-25]. Herein, a new xenograft cell line derived from malignant cystosarcoma phyllodes of the human breast is described.

Clinical Data

A needle biopsy of a mass in the right breast of a 34-year old woman in 1992 was consistent with a diagnosis of cystosarcoma phyllodes. The mastectomy specimen consisted of four fragments of tissue measuring 4×2.5×2 cm for the largest one and a diameter of 1 cm for smaller ones. The largest fragment revealed a soft and yellowish lobule in the cut surfaces. Histologically, the tumor was composed of ductal cells, myoepithelial cells and stromal elements. A recurrence of a nodule in the surgical scar in June 1999 occurred followed by rapid acceleration of growth. The patient was subjected to further resection (including two ribs) with flap reconstruction in June 1999. The specimen had been partially bisected showing a solid grayish circumscribed non-encapsulated tumor measuring 5×5×3 cm with foci of hemorrhage, myxoid change and necrosis. The tumor was composed of interlacing fascicles of spindle cells with no epithelial component. One lymph node with no tumor involvement was identified. The patient developed a second relapse in February 2000, with a diagnosis being metastatic sarcomatoid neoplasm and consistent with recurrent malignant cystosarcoma phyllodes. The tumor proved not responsive to either radiation or chemotherapy; local excision was performed on Mar. 17, 2000. A computerized tomographic (CT) scan of the thorax on Feb. 22, 2000 revealed a 5.5×4.5×5.6 cm sternal mass and a 5 mm lung nodule in the right upper lobe. Quite extensive areas of necrosis were shown by the cut surfaces of the specimen tumor, while sectioning revealed destruction of the sternum and invasion into soft tissue in front of the sternum. Positron Emission Tomography (PET) scan was solely performed as a baseline for evaluation. All methods, including alternative medicine proved unsuccessful.

Xenografting

For tumor transplant or xenografting, a portion of minced clinical tumor was inoculated subcutaneously into the flanks of nude mice.

The tumor transplant was observed to develop palpable tumor by angiogenesis. There was a latency period of approximately two months before the implanted tumors began to grow. The growth rate was slow, with a doubling time of longer than 15 days. The tumor was then aseptically removed after 25 days. A portion of which was re-implanted into another female nude mouse to study the in vivo growth characteristics and the tumorigenic potential. The tumors from the first and subsequent xenografting grew rapidly and required transplantation monthly. The tumor line was then grown and maintained through serial animal.

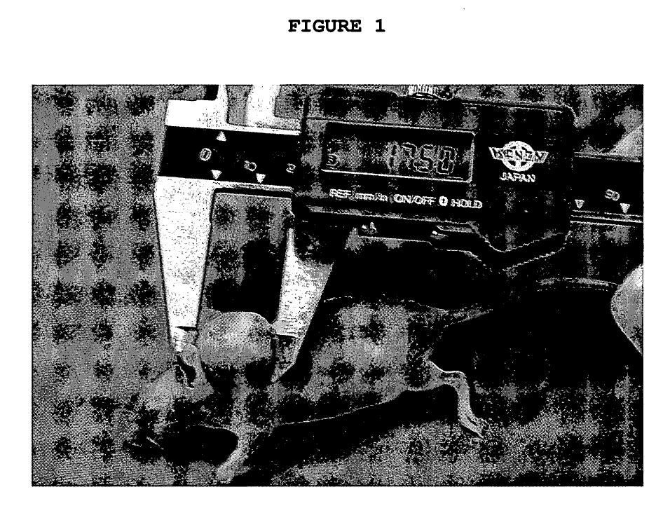

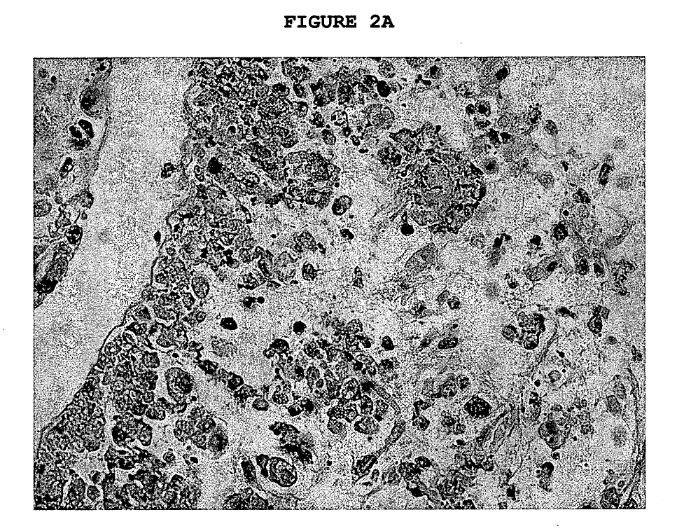

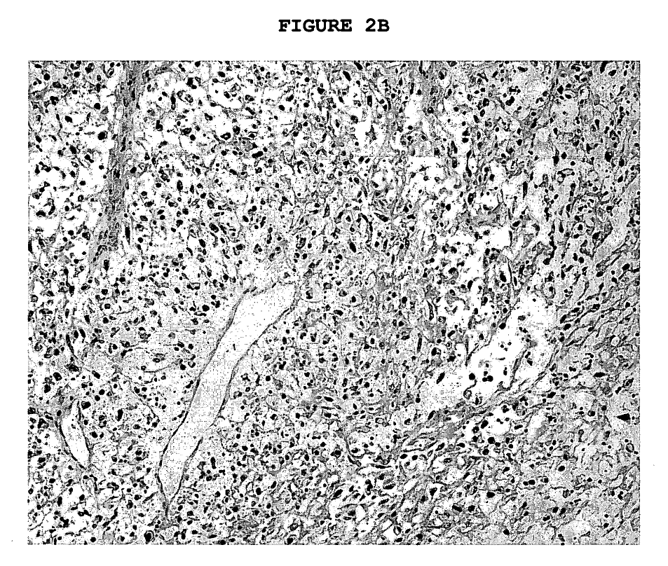

A portion of the primary tumor and the xenograft were placed into tissue cultures. The cell line originated from the xenograft showed tumorigenicity in nude mice. One cell line-derived tumor grown in a nude mouse for 3 weeks (FIG. 1) attained a weight of 5-6 g on removal, with volume being 30 mm³. Histologically, the tumors derived from the cell line appeared similar to the transplantable tumor (FIG. 2).

Establishment of the MCP-5 Cell Line

The primary tumor surgically removed from the patient and the xenografts were minced finely into pieces of≦1 mm³ using two sterile scalpels. They were distributed into a plastic 75 cm² cell culture flask (Corning, N.Y., USA), with D-MEM/F-12 (Dulbecco's Modified Eagle Medium with 15% Fetal Bovine Serum (FBS), 1 mM HEPES buffer, L-glutamine, pyridoxine hydrochloride, 100 U/ml penicillin, 100 μg/ml streptomycin and 4 μg/ml insulin) (Invitrogen Corporation, USA). The culture flask was incubated at 37° C. in 95% air and 5% CO₂. All tissue culture medium was removed and replaced with fresh medium every 3 days.

Once the MCP-5 cell line was established, samples of the monolayer cells in the flask were treated with 0.25% trypsin and 1 mM ethylene diamine tetraacetic acid (Invitrogen Corporation, USA) for 3-5 minutes, followed by neutralization with culture medium containing FBS. The cells were re-suspended in 10 ml culture medium containing 15% FBS after centrifugation. The sub-cultivation ratio was 1:10 whereas the rest of the cells would be immersed in 5% dimethyl sulfoxide (DMSO) and 95% FBS before being preserved in liquid nitrogen at −196° C. for storage.

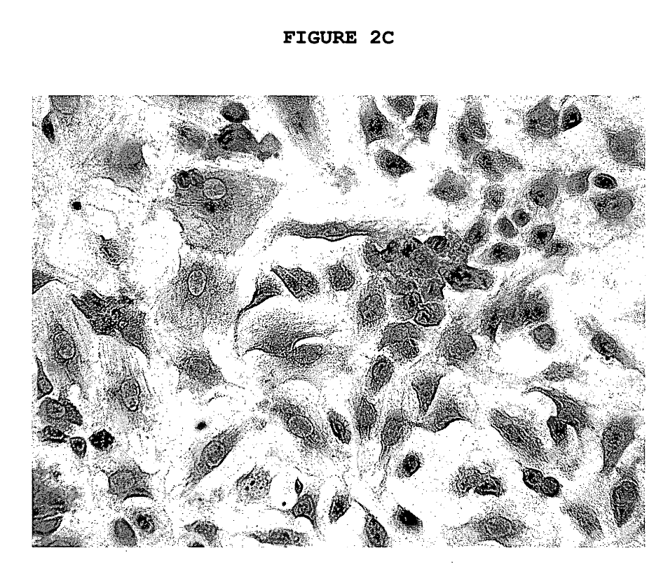

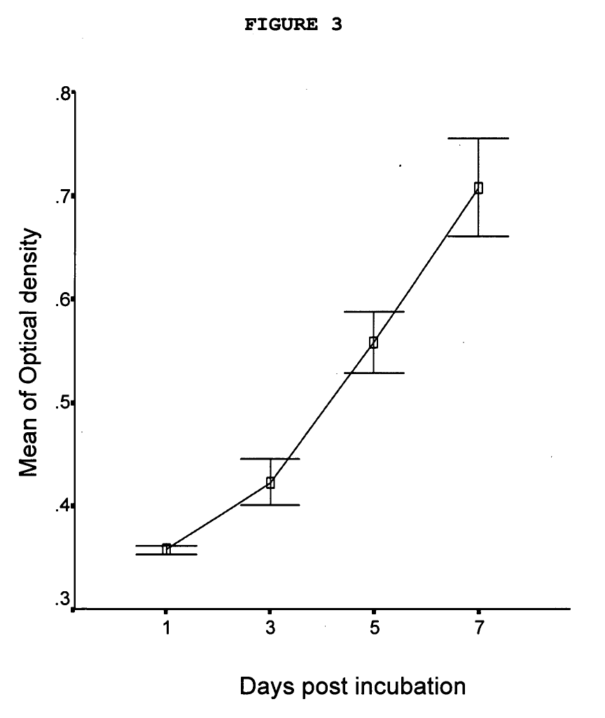

The growth of these cells was investigated in terms of rate of proliferation by the Universal Microplate Reader (Bio-Tek Instruments, Inc. USA). The cultures from the mastectomy tissue showed no growth and therefore were discarded. On the other hand, the cultures derived from passage of the transplantable tumor in nude mice showed sustained growth of attached plump pleomorphic spindle-shape cells. These cells grow stably and slowly in monolayer in the pattern of malignant cells with spindle cells crossing one another and with partial loss of adhesion at high density. Cells (1.0×10⁵/ml) grew to confluence with fairly uniform growth from the day of passage to harvest day and subculture onto new 75 cm² flask was required once weekly. The growth of these MCP-5 cells was in a lag phase in the initial 24 hours, followed by a logarithmic phase during the first and fourth days. The cells entered the stationary phase at the fifth day. The proliferation rate is shown in FIG. 3.

Immunohistochemistry

Various antigens (antibodies were obtained from Dako Corporation, Carpinteria, Calif.) namely cytokeratin (CK), desmin, Ewing's sarcoma marker, estrogen receptor (ER), laminin-binding receptor (LBR), mucin 1 (MUC1), progesterone receptor (PR), sarcomeric actin, and vimentin, were immuno-cytochemically tested. This is performed by using a labeled streptavidin biotin (LSAB) complex kit (Dako Corporation, Carpinteria, Calif.). Monolayers of cells cultured on glass coverslips (Menzel-Glaser, Germany) were washed three times in 0.01 M phosphorsaline buffer (PBS). Cells were fixed in 4% paraformaldehyde overnight at 4° C. To block nonspecific antibody binding sites, cells were treated with 0.3% H₂O₂ for 20 minutes, followed by 0.8% horse serum for 20 minutes at room temperature, then incubated with primary antibodies (Dako Corporation, Carpinteria, Calif.) at dilutions of 1:100 at 4° C. overnight or 37° C. for an hour.

The same procedures were applied for the paraffin sectioning except for the sections which were primarily de-waxed and treated with 3% H₂O₂ in methanol for 10 minutes. Antigen retrieval was performed in 10 mM citrate buffer at pH 6.0 and microwaved prior to cooling down at room temperature. Incubation with primary antibodies was preceded by treatment for blocking nonspecific antibody binding sites with 10% horse serum for at least 30 minutes.

Cells and sections were washed extensively with PBS after incubation, and treated with biotinylated link for 45 minutes and streptavidin-HRP for 45 minutes. Staining was performed using 2% DAB substrate-chromogen solution for 10 minutes. Hematoxylin was used as a nuclear counterstain. The sections were dehydrated, cleared, and mounted with synthetic resin.

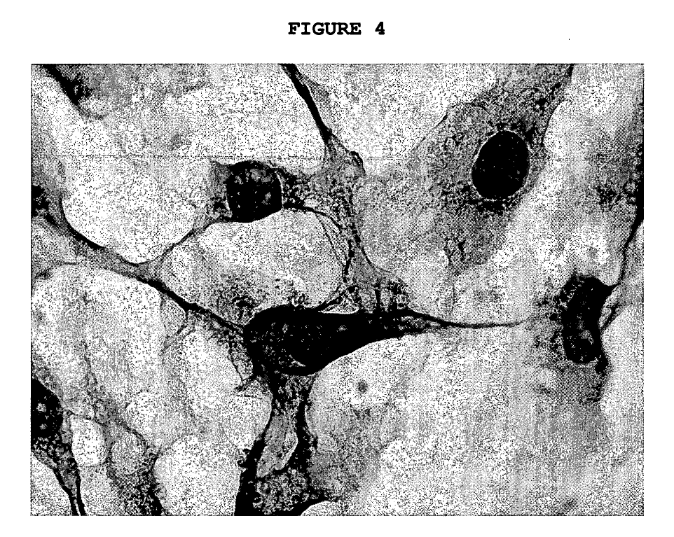

Immunohistochemistry was performed on the nude mouse solid tumor as well as on MCP-5 xenograft cell line and the results are shown in Table 1. The intensity of the stain was graded as negative (−), weak (+), or strong (++), depending on the number of cells stained and the darkness of the DAB precipitate. These cells are clearly illustrated to be hyperchromatic with VEGF (FIG. 4).

TABLE 1
Antigen characteristics of nude mouse
solid tumor and xenograft cell line.

| Antibodies | original solid tumor | Xenograft |
|---|---|---|
| Cytokeratin | + | + |
| Oestrogen receptor | − | + |
| Progesterone receptor | − | ++ |
| Vimentin | + | + |
| VEGF | + | ++ |

The MCP-5 cells were positive for cytoplasmic immunoreactivity of cytokeratin (CK) and mucin 1 (MUC1). Cytokeratin 19 is an intermediate filament protein expressed by normal and malignant mammary cells in addition to other epithelial cells, myoepithelial cells and derived malignancies [31-33]. Concurrent measurements of cytokeratin expression served to identify tumor cells of epithelial origin [34].

Increased expression of the epithelial mucin1 has been linked to tumor aggressiveness in human breast carcinoma [35].

On the other hand, vimentin and Ewing's sarcoma marker were both negatively stained. Vimentin is the intermediate filament detected in mesenchymal cells, including fibroblasts and smooth-muscle cells [8]. This had inspired us to engage the use of vimentin for the confirmation of its absence.

Desmin and laminin-binding receptor were strongly positively shown on tissue sections as to delineate the presence of those membrane markers. Neoplastic cells in formalin-fixed, paraffin-embedded tissue sections which were found to be positive for desmin were attributed to the attendance of vessel walls [36, 37]. Desmin is a filament that is present in smooth-muscle cells [38]. However, desmin was negative in all tumor cells except for skeletal muscle cells in Wilms' tumor [39].

Laminin is one of the major components of the basement membrane. Laminin receptors are thought to be involved in a wide variety of biological events including development of tumor invasion and metastasis through cell-cell and/or cell-extracellular matrix interactions [40]. It was therefore, reasonable for the cell line to show negative staining since cultured cells lacked a basement membrane i.e. supporting structures. These cells showed positive immunostaining for mucin and sarcomeric actin. Peter et al. formerly validated that negative staining for sarcomeric actin indicates an absence of endothelial cells [41]. Actin is a filament that is present in smooth-muscle cells as well as myoepithelial cells [42].

The phyllodes tumor cells demonstrated negatively with estrogen but positively with progesterone, even though estrogen receptors and progesterone receptors had previously been reported in phyllodes tumors [43-47] and had been suggested as the basis for initiating treatment. Alberti et al. also stated that most of their specimens were either completely ER negative or displayed low titers of specific estrogen binding [5].

Three conventional breast cancer cell lines have been employed for comparison with MCP-5, MDA-MB-231 and MCF-7, and showed only intermediate expression of MUC1 whereas T47D revealed abundant expression. Low levels of MUC1 expression are associated with decreased expression of cytokeratin and increased expression of vimentin [48]. MDA-MB-231 cells have a high proliferation rate, thereby expressing the intermediate filament vimentin but lacking estrogen receptors [49, 50]. MCF-7 and T47D cells, in contrast, are ER positive [51] but vimentin was negatively stained [52]. PR localization was investigated with the PR-rich T47D cells [53]. When MCF-7 cells were grown, the PR synthesis increased significantly [54].

TABLE 2
Comparison of expression of the markers
of UHKBR-01 with MCF-7, MDA-MB231 and T47D.

| Antibodies | MDA-MB-231 | MCF-7 | T47D |
|---|---|---|---|
| Cytokeratin | + | + | + |
| Vimentin | + | − | − |
| Oestrogen receptor | − | + | + |
| Progesterone | − | + | + |
| Mucin 1 | ++ | ++ | +++ |

Histologically, p53 and vascular epithelial growth factor (VEGF) were positively stained, though not intense. The elevated cellular content of p53 is a common event in invasive palpable mammary tumors [55]. The role of p53 in tumorigenesis is not only confined to mammary tumor in human but also in animal (canine) [56].

Angiogenesis induction paralleled VEGF production by the tumor cells [57]. Callgy et al. have also suggested that measurement of tumor VEGF, as an indicator of angiogenesis, is reliable prognostically [58].

Light and Electron Microscopy

Monolayers of cultured cells or tumor tissues from nude mice were fixed in 10% formalin and processed for sectioning and hematoxylin staining. For electron microscopy, 1-mm cubes of tumor tissue or monolayers of culture cells were fixed by modified Karnovsky's fixative (2.5% glutaraldehyde, 2% paraformaldehyde in 0.1M sodium cacodylate buffer, pH 7.4). Samples were fixed at 4° C. for 4 to 12 hours. Material was then rinsed briefly in buffer, post fixed in 1% buffered osmium tetroxide, and dehydrated in graded ethanol. Specimens were then rinsed in propylene oxide and infiltrated and embedded in Araldite resin. Representative sections were prepared using an LKB Ultratome III. Thick sections (1 μ) were stained with toluidine clue or Ladd's multiple stains (Paragon Stain, Ladd Research Industries, Burlington, Vt.). Thin sections (800 Å) were stained with aqueous urangyl acetate, counterstained with Reynolds' lead citrate, and examined in an RCA EMU-4 electron microscope (RCA Corp., Cherry Hill, N.J.).

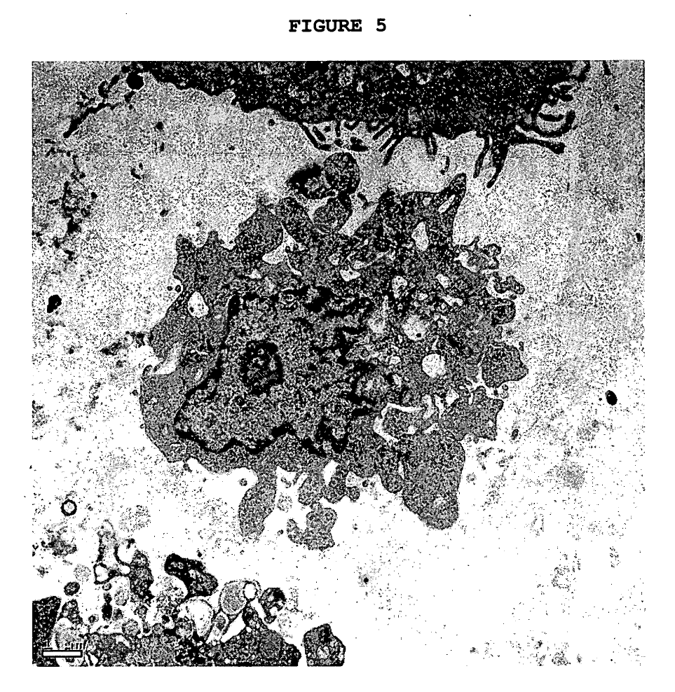

The epithelial component of the monolayer cultured cells (FIG. 5) showed ducts lined by one or two layers of columnar cells exhibiting intercellular desmosomes and apical microvilli projecting within the duct lumen. A single layer of myoepithelial cells surrounded the ductal epithelium. The stromal cells consisted of a variable mixture of fibroblasts and myofibroblasts. The fibroblasts wherein were characterized by numerous dilated rough endoplasmic reticular, whereas the myofibroblasts showed in addition peripheral intermediate filaments and a few dense bodies.

Another stromal component showed elongated spindle cells surrounded by basal lamina. The cytoplasm contained abundant intermediate filaments interspersed with dense bodies. Pinocytotic vesicles and dense plaques were seen on the cell surface. The features are characteristic of smooth-muscle differentiation.

Yet another stromal component was formed by single or small groups of spindle cells joined by occasional tight junctions or desmosomes. The cytoplasm showed fine filaments with dense bodies at the periphery, as well as aggregates of tonofilaments around the nucleus. These features are those of myoepithelial.

The cells and the nuclei display pleomorphism. Characteristically, the nuclei contain an abundance of DNA and are extremely dark staining (hyperchromatic). The nuclei are disproportionately large for the cell, and the nuclear-to-cytoplasmic ratio may approach 1:1 instead of the normal 1:4 or 1:6. The nuclear shape is usually extremely variable, and the chromatin is often coarsely clumped and distributed along the nuclear membrane. Large nucleoli are usually present in these nuclei [59].

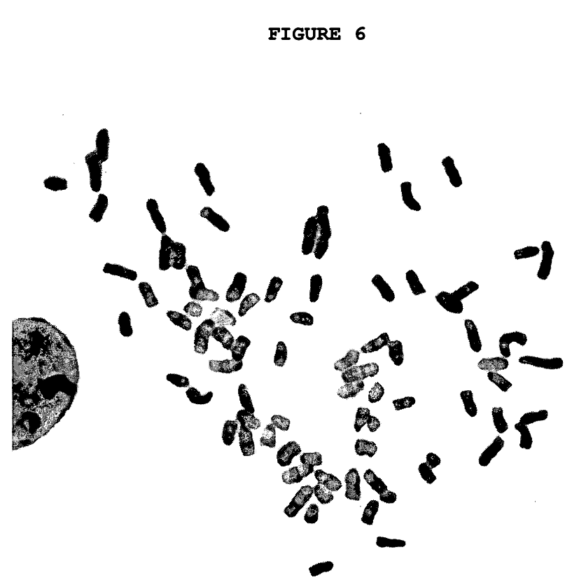

Karyotype analysis was then performed in a cell line originating from the xenograft. FIG. 6 revealed that the cultured cells were of mouse origin. For virus-like particle measurement, the cells were cultured in the same conditions as described earlier. They were then harvested and centrifuged for 5 minutes to separate the cells. The cell culture supernatant was then transferred into 3×1.5 ml Eppendorf tubes, and then ultracentrifuged at 19500 rpm (˜30000 g) for 4 hours using the Avanti Centrifuge with adaptors placed inside the carousel (Beckham). The supernatant was then aspirated to transfer all the sediment to a single Eppendorf tube. The sediment was then placed in a cassette and stained for electron microscopy.

Karyotyping proved this cell line to be from mouse origin rather than human. The study design obviously rules out chemical agents as a causative factor. The absence of virus-like particles in the cell culture seems to indicate that a mice minute parvovirus, which has been shown previously to result in DNA transfer [60] between cells of different biological origin, is not responsible for this unusual turn of events. One possibility is a mutation has occurred during tumorigenesis; however, it may be that some other unknown mechanism, which may be unique to this type of tumor, may be accountable.

These findings led to the conclusion that these cells being highly metabolic and malignant in nature. Moreover, the MCP-5 cell line serves as a cystosarcoma phyllode model for further investigation on the pathogenesis and therapy of this rare cancer.

### REFERENCES

1. John Hart, Lester J. Layfield, William E. Trumbull, Donald Brayton, Wiley F. Barker, Armando E. Guliano, “Practical Aspects In The Diagnosis And Management Of Cystosarcoma Phyllodes,” Arch. Surg. 123, 1079-1083, 1988.
2. Darryl Carter, “Interpretation Of Breast Biopsies,” Chapter 9, Third Ed. Philadelphia, N.Y., 1996.
3. Prabha, B. Rajan, Milicent, L. Cranor, and Paul Peter Rosen, “Cystosarcoma Phyllodes In Adolescent Girls And Young Women,” Am. J. Surg. Pathlo. 22(1): 64-69, 1998.
4. Christensen, L., Nielsen, M., Madsen, P. M., “Cystosarcoma Phyllodes—A Review Of 19 Cases With Emphasis On The Occurrence Of Associated Breast Carcinoma,” Acta. Path. Microbiol. Immunol. Scand., Sect. A, 94:443-5, 1986.
5. Alberti, O Jr, Brentani, M. M., Goes, J. C. S., Lemos, L. B., Torioni, H., “Carcinoembryonic Antigen—A Possible Predictor Of Recurrence In Cystosarcoma Phyllodes,” Cancer, 57: 1042-5, 1986.
6. Anderson, A., Bergdahl, L., “Cystosarcoma Phyllodes in Young Women,” Arch. Surg., 113: 742-4, 1987.
7. Michael, A. Warso, Rajeshwari, R. Mehta, Gloria, D. Hart, Jewell, M. Graves and Albert Green, “A Cell Line Derived From A Clinically Benign Phyllodes Tumor: Characterization and Implication,” Anticancer Research 15, 399-404, 1995.
8. Manon Auger, Wedad Hanna, Harriette J. Kahn, “Cystosarcoma Phyllodes Of The Breast And Its Mimics,” Arch. Pathlo. Lab. Med., 113,1231-1235, 1989.
9. Chua, C. L., Thomas, A. and Ng, B. K., “Cystosarcoma Phyllodes—Asian Variations,” Aust. N.Z.J. Surg., 58, 301-305, 1988.
10. Muller, J. Ueber den feinern Bau and Formen krankhaften Geschwuelste, Berlin: G. Reimer, p. 54, 1838.
11. Lee, B. and Pack, G. “Giant Intracanalicular Fibroadenomyxoma Of The Breast,” Am. J. Cancer, 15, 2583-2609, 1931.
12. Halverson, J. D. and Hori-Rubaina, J. M., “Cystosarcoma Phyllodes Of The Breast,” Amer. Surg., 40, 295-301, 1974.
13. Pietruszka, M. And Barnes, L., “Cystosarcoma Phyllodes: A Clinico-Pathologic Analysis Of 42 Cases,” Cancer, 41, 1974-83, 1978.
14. Norris, H. J. and Taylor, H. B., “The Relationship Of Histologic Features To Behaviors Of Cystosacoma Phyllodes: Analysis Of Ninety-Four Cases,” Cancer, 20, 2090-9, 1967.
15. Kessinger, A., Foley, J. F., Lemon, H. M., Metastatic Cystosarcoma Phyllodes: A Case Report And Review Of The Literature,” J. Surg. Oncol., 4, 131-147, 1972.
16. Hajdu, S. I., Espinosa, M. H., Robbins, G. F., “Recurrent Cystosarcoma Phyllodes. A Clinico-Pathologic Study Of 32 Cases,” Cancer, 38, 1402-1406, 1976.
17. Blichert-Toft, M., Hansen, J. P. H., Hasen, O. H. and Schiodt, T., “Clinical Course Of Cystosarcoma Phyllodes Related To Histologic Appearance,” Surg. Gynecol. Obstet., 140, 929-932, 1975.
18. Contarini, O., Urdaneta, L. F., Hagan, W. and Stephenson, S. E., “Cystosarcoma Phyllodes Of The Breast: A New Therapeutic Proposal,” Am. Surg., 48, 157-166, 1982.
19. Macejova, D., Brtko, J., “Chemically Induced Carcinogenesis: A Comparison Of 1-Methyl-1-Nitrosourea, 7,12-Dimethylbenzanthracene, Diethylnitroso-Amine And Azoxymethan Models (Minireview),” Endocr. Regul., 35(1): 53-9, 2001.
20. Ward, R. M., Evans, H. L., “Cystosarcoma Phyllodes: A Clinicopathologic Study Of 26 Cases,” Cancer 58:2282-2289, 1986.
21. Murad, T. M., Hines, J. R., Beal, J., Bauer, K., “Histopathologic And Clinical Correlations Of Cystosarcoma Phyllodes,” Arch. Pathol. Lab. Med. 112: 752-756, 1988.
22. Vorherr, H., Vorherr, U. F., Kutvirt, D. M., Key, C. R., “Cystosarcoma Phyllodes: Epidemiology, Pathohistology, Pathobiology, Diagnosis, Therapy, And Survival,” Arch. Gynecol. 236:173-181, 1985.
23. Bruno Salvadora, Fabio Cusumano, Romualdo del Bo, Vincenzo Delledonne, Massimo Grassi, Dario Rovini, Roberto Saccozzi, Salvatore Andreola and Claudio Clemente, “Surgical Treatment Of Phyllodes Tumors Of The Breast,” Cancer 63:2532-2536, 1989.
24. Lewko, W. M., Vaghmar, R., Malcchar, J. R., Husseini, S., Montgomery, C. A., Thurman, G. B., et al., “Cultured Breast Custosarcoma Phyllodes Cells And Applications To Patient Therapy,” Breast Cancer Res. Treat. 17:131-138, 1990.
25. Tibbetts, L. M., Poisson, M. H., Tibbetts, L. L., Cummings, E. J., “A Human Breast Stromal Sarcoma Cell Line With Features Of Malignant Cystosarcoma Phyllodes,” Cancer 62: 2176-2182, 1988.
26. Siwek, B., Larsimont, D., Lacroix, M., Body, J. J., “Establishment And Characterization Of Three New Breast-Cancer Cell Lines,” Int. J. Cancer, 76(5) 677-683, 1998.
27. Watanabe, M., Tanaka, H., Kamada, M., Okano, J. H., Takahashi, H., Uchida, K., Iwamura, A., Zeniya, M., Ohno, T., “Establishment Of The Human BSMZ Breast Cancer Cell Line Which Overexpresses The Erbb-2 And C-Myc Genes,” Cancer Res, 52(19):5178-5182.
28. Tokuda, Y., Kubota, M., Ueyama, Y. et al., “A Malignant Cystosarcoma Phyllodes With Positive Oestrogen Receptor And Its Heterotransplantation,” Cancer 55: 370-374, 1984.
29. Kahn, H. J., Baumal, R., From, L., “Role Of Immunohistochemistry In The Diagnosis Of Undifferentiated Tumors Involving The Skin,” J. Am. Acad. Dermatol., 14: 1063-1072, 1986.
30. Baumal, R., Kah, H. J., Bailey, D., “The Value Of Immunohistochemistry In Increasing Diagnostic Precision Of Undifferentiated Tumors By The Surgical Pathologist,” Histochem. J., 16:1061-1078, 1984.
31. Osborn, M., Weber, K., “Tumor Diagnosis By Intermediate Filament Typing: A Novel Tool For Surgical Pathology,” Lab. Invest., 48: 372-394, 1983.
32. Fuchs Em Weber K., “Intermediate Filaments: Structure, Dynamics, Function, And Disease,” Ann. Rev. Biochem., 63, 345-82, 1994.
33. Franke, W. W., Schmid, E., Freudenstein, C., et al., “Intermediate Sized Filaments Of The Prekeratin Type In Myoepithelial Cells,” J. Cell. Biol., 84: 633-654, 1980.
34. Sundström, B. E., Stigbrand, T. I., “Cytokeratins And Tissue Polypeptide Antigen,” Int. J. Biol. Sci., 9(2), 102-108, 1994.
35. Porowska, H., Paszkiewicz-Gadek, A., Wolczynski, S., Gindzieski, A., “MUC1 Expression In Human Breast Cancer Cells Is Altered By The Factors Affecting Cell Proliferation,” Neoplasma., 49(2):104-109, 2002.
36. Katrin Neubauer, Thomas Knittel, Sabine Aurisch, Peter Fellmer and Giuliano Ramadori, “Glial Fibrillary Acidic Protein—a cell type specific marker for Ito cells in vivo and in vitro,” J. Hepatol., 24(6), 719-730, 1996.
37. Lae, Marick E, Roche, Patrick C, Jin, Long, Lloyd, Ricardo V, Nascimento, Antonio G., “Desmoplastic Small Round Cell Tumor: A Clinicopathologic, Immunohistochemical, And Molecular Study Of 32 Tumors,” The Am. J. Surg. Pathol., 26(7), 823-835, 2002.
38. Debus, E., Weber, K., Osborn, M., “Monoclonal Antibodies To Desmin, The Muscle Specific Intermediate Filament Protein,” Embo. J., 2: 2305-2312, 1983.
39. Ogawa,K., Nakashima,Y., Yamabe,H., Hamashima,Y., “Clear Cell Sarcoma Of The Kidney An Immunohistochemical Study,” Acta Pathologica Japonica, 36(5), 681-689.23, 1986.
40. I.Ozaki, K.Yamamoto, T.Mizuta, S.Kajihara, N.Fukushima, Y.Setoguchi, F.Morito, T.Sakai, “Differential Expression Of Laminin Receptors In Human Hepatocellular Carcinoma,” Gut, 43: 837-842, 1998.
41. Peter, M. G., Davenport, A. P., “Delineation Of Endothelin Receptors In Human Left Ventricular Smooth-Muscle Cells,” J. Cardiovasc. Pharm., 26(3): S355-S357, 1995.
42. Tsukada, T., McNutt, M. A., Ross, R., Gown, A. M., “HHF 35, A Muscle Actin-Specific Monoclonal Antibody, II: Reactivity In Normal, Reactive Neoplastic Human Tissues,” Am. J. Pathol., 127: 389-402, 1987.
43. Brentani, M. M., Nagai, M. A., Oshima, C., Pacheco, M. M., Goes, J. C. S., Lemos, L. B., “Steroid Receptors In Cystosarcoma Phyllodes,” Cancer Detect. Prev. 5: 211-219, 1982.
44. Rao, B. R., Meyer, J. S., Fry, C. G., “Most Cystosarcoma Phyllodes And Fibroadenomas Have Progesterone Receptor But Lack Estrogen Receptor:

Stromal Localization Of Progesterone Receptor,” Cancer 47(8): 2016-2021, 1981.
45. Palshof, T., Blichert-Toft, M., Daehnfeldt, J. L., “Estradiol Binding Protein In Cystosarcoma Phyllodes Of The Breast,” Eur. J. Cancer, 16: 591-593, 1980.
46. Tokuda, Y., Kubota, M., Ueyama, Y., “A Malignant Cystosarcoma Phyllodes With Positive Esstrogen Receptor And Its Hetero-Transplantation Into Nude Mice,” Cancer 55: 370-374, 1985.
47. Porton, W. M., Poortman, J. “Estrogen Receptors In Cystosarcoma Phyllodes Of The Breast,” Eur. J. Cancer, 17: 1147-1149, 1981.
48. Walsh, M. D., “Heterogeneity Of MUC1 Expression By Human Breast Carcinoma Cell Lines In Vivo And In Vitro,” Breast Cancer Res. Treat., 58:255-66, 1999.
49. Hardwick, M., “Peripheral-Type Benzodiazepine Receptor Levels Correlate With The Ability Of Human Breast Cancer MDA-MB-231 Cell Line To Grow In SCID Mice,” Int. J. Cancer, 94:322-7, 2001.
50. Maemura, M., “Expression And Ligand Binding Of Alpha 2 Beta 1 Integrin On Breast Carcinoma Cells,” Clin. Exp. Metastasis, 13:223-35, 1995.
51. Cao, S., Hudnall, S. D., “Measurement Of Estrogen Receptors In Intact Cells By Flow Cytometry,” Cytometry 4:109-14, 2000.
52. Pieper, F. R., “Regulation Of Vimentin Expression In Cultured Epithelial Cells,” Eur. J. Biochem., 210:509-19, 1992.
53. Elashry-Stowers, D., “Immunocytochemical Localization Of Progesterone Receptors In Breast Cancer With Anti-Human Receptor Monoclonal Antibodies,” Cancer Res., 48:6462-74, 1988.
54. Parikh, I., “Are Estrogen Receptors Cytoplasmic Or Nuclear? Some Immunocytochemical And Biochemical Studies,” J. Steroid. Biochem., 27:185-92, 1987.
55. Soussi,T., “The P53 Tumor Suppressor Gene: From Molecular Biology To Clinical Investigation,” Ann. N. Y. Acad. Sci.; 910:121-37, discussion 137-9, 2000.
56. Tokino,T., “Tumor Suppressor Gene P53,” Nippon Rinsho, 54(4), 960-4, 1996.
57. Lichtenbeld, H. C., Barendsz-Janson, A. F., van Essen, H., Struijker Boudier, H., Griffioen, A. W., Hillen, H. F., “Angiogenic Potential Of Malignant And Non-Malignant Human Breast Tissues In An In Vivo Angiogenesis Model,” Int. J. Cancer; 77(3), 455-9, 1998.
58. Callagy, G., Dimitriadis, E., Harmey, J., Bouchier-Hayes, D., Leader, M., Kay, E. “Immunohistochemical Measurement Of Tumor Vascular Endothelial Growth Factor In Breast Cancer. A More Reliable Predictor Of Tumor Stage Than Microvessel Density Or Serum Vascular Endothelial Growth Factor,” Appl. Immunohistochem. Mol. Morphol., 8(2), 104-9, 1999.
59. Cotran, Kumar, Collins, Robbins, “Pathologic Basis Of Disease,” Sixth Ed., Chapter 8, p. 260, 2000.
60. Hernando E. et al., “Biochemical And Physical Characterization Of Parvovirus Minute Virus Of Mice Virus-Like Particles,” Virology 267:299-309, 2000.
61. Khan, S. A., Badve, S., “Phyllodes Tumors Of The Breast,” Curr. Treat. Options Oncol., 2(2):139-47, 2001.
62. Paulsen, F., Belka, C., Gromoll, C., Hehr, T., Friedel, G., Wolf, H., Budach, W., Bamberg, M., “Cystosarcoma Phyllodes Malignum: A Case Report Of A Successive Triple Modality Treatment,” Int. J. Hyperthermia, 16(4):319-24, 2000.
63. Pandey, M., Mathew, A., Kattoor, J., Abraham, E. K., Mathew, B. S., Rajan, B., Nair, K. M., “Malignant Phyllodes Tumor,” Breast J., 7(6):411-6, 2001.
64. Parker, S. J., Harries, S. A., “Phyllodes Tumors,” Postgrad. Med. J., 77(909):428-35, 2001.

What is claimed is:

1. A cell line of mouse origin derived from a human malignant cystosarcoma phyllodes tumor designated MCP-5.

2. A method for establishing a cell line of mouse origin derived from a human malignant cystosarcoma phyllodes tumor, comprising:
a. transplanting the human malignant cystosarcoma phyllodes tumor to a recipient nude mouse;
b. removing the tumor from the recipient nude mouse after allowing sufficient time for tumor growth; and
c. culturing the cells of the tumor in a suitable culture medium, thereby establishing a cell line of mouse origin derived from a human malignant cystosarcoma phyllodes tumor.

3. The method according to claim 2, wherein the tumor is transplanted using subcutaneous inoculation.

4. The method according to claim 2, wherein the suitable culture medium is D-MEM/F-12.
